(19) United States
(12) Patent Application Publication
(10) Pub. No.: US 2013/0237819 A1
(43) Pub. Date: Sep. 12, 2013

(54) CONTROLLER FOR CONTROLLING OPERATIONS OF AN ULTRASONIC DIAGNOSIS DETECTOR

(71) Applicants: Jie SUN, Wu Xi (CN); Ningmei Liu, Wu Xi (CN); Xingjie Feng, Wu Xi (CN); Qishun Gu, Wu Xi (CN); Dao Zhou, Wu Xi (CN); Jianguo Zheng, Wu Xi (CN); Yafang Cheng, Beijing (CN)

(72) Inventors: Jie SUN, Wu Xi (CN); Ningmei Liu, Wu Xi (CN); Xingjie Feng, Wu Xi (CN); Qishun Gu, Wu Xi (CN); Dao Zhou, Wu Xi (CN); Jianguo Zheng, Wu Xi (CN); Yafang Cheng, Beijing (CN)

(21) Appl. No.: 13/601,852

(22) Filed: Aug. 31, 2012

(30) Foreign Application Priority Data
Aug. 31, 2011 (CN) 201110270190.5

(51) Int. Cl. A61B 8/00

(52) U.S. Cl. CPC A61B 8/54
USPC 600/437; 600/407

(57) ABSTRACT

A controller comprising one or more control units, one or more triggering components arranged on the one or more control units, wherein the one or more triggering components are used for triggering the one or more control units to generate a corresponding control instruction, thereby controlling operations of the ultrasonic diagnosis detector, wherein at least one of the one or more control units is configured to be fixed on a portion of a body and/or a probe of the ultrasonic diagnosis detector.

### BACKGROUND OF THE INVENTION

1. Field of the Invention

Embodiments of the present invention relate to a controller, and, more particularly, to a controller for controlling operations of an ultrasonic diagnosis detector.

2. Description of the Prior Art

With the development of the medical technology, ultrasonic diagnosis detectors are widely applied to the current medical diagnosis. When operating an ultrasonic diagnosis detector, a user operates a host machine of the ultrasonic diagnosis detector by means of a console so as to emit ultrasonic wave through a probe and display an image generated from an echo signal of the ultrasonic wave on a display.

In the traditional application, a user needs to control the position of a probe with one hand and control operations of an ultrasonic diagnosis detector on the main operating console with the other hand, for example, freezing an image, scaling an image, adjusting scanning depth, etc. As such, since a user is required to operate the ultrasonic diagnosis detector with both hands and observe the display at the same time, it is not convenient to perform operations. In practice, a user always keeps a relatively long distance from the main console and the host machine, which further enhances the inconvenience of operations.

When a user uses a conventional diagnosis detector equipped with a wireless control device, the user still needs to operate a probe with one hand and the wireless control device with the other hand. Thus, operations with one single hand is impossible, and thereby no hand can he freed to perform other operations so as to improve the usability and diagnostic efficiency of instruments.

In addition, it is suggested that control buttons be integrated with a probe of an ultrasonic diagnosis detector. However, the integrated probe needs manufacturing integrally. Even if just a control button is damaged, the whole probe needs to be replaced. Meanwhile, the addition of control buttons will change the shape and the whole production process of the probe Moreover, one needs to design and produce different probes for users having different operation habits (such as for left-handed users).

Thus, a controller, which is flexible to use, simple to manufacture, and convenient to replace is desired to control operations of an ultrasonic diagnosis detector.

### BRIEF SUMMARY OF THE INVENTION

In one embodiment of the invention, a controller for controlling operations of an ultrasonic diagnosis detector is provided. The controller comprises one or more control units and one or more triggering components arranged on the one or more control units. The one or more triggering components is/are configured to trigger the one or more control units to generate a corresponding control instruction to control operations of the ultrasonic diagnosis detector. At least one of the one or more control units is configured to be fixed on a portion of a body and/or a probe of the ultrasonic diagnosis detector.

In another embodiment of the invention, an ultrasonic diagnosis detector is provided. The detector comprises an ultrasonic diagnosis detector host machine, a probe and a console configured to control the ultrasonic diagnosis detector host machine so as to emit ultrasonic wave through the probe. The ultrasonic diagnosis detector host machine generates an image based on an echo signal of the ultrasonic wave. The detector further comprises a display configured to display the image, and a controller configured to control operations of the ultrasonic diagnosis detector, wherein the controller is configured to be fixed on a portion of a body and/or the probe.

In yet another embodiment of the invention, a controller is provided. The controller comprises one or more control units, and one or more triggering components arranged on the one or more control units. The one or more triggering components is/are configured to trigger the one or more control units to generate a corresponding control instruction, wherein at least one of the one or more control units is configured to be fixed on a portion of a body.

### BRIEF DESCRIPTION OF THE DRAWINGS

The advantages, features, and characteristics of the present invention will be further understood through the following depiction of the embodiments of the present invention in combination with the drawings.

FIG. 1 shows an ultrasonic diagnosis detector according to an embodiment of the present invention;

FIG. 2 shows a controller according to an embodiment of the present invention;

FIG. 3 shows the status where control units of a controller are fixed on fingers of a user according to an embodiment of the present invention;

FIG. 4 shows the circuit structure of a control unit of a controller according to an embodiment of the present invention,

FIG. 5 shows the circuit structure of a wireless unit on a host machine of an ultrasonic diagnosis detector according to an embodiment of the present invention;

FIG. 6 shows how to charge control units of a controller according to an embodiment of the present invention;

FIG. 7 shows the unfolding status of a control unit of a controller according to an embodiment of the present invention;

FIG. 8 shows the status where control units of a controller are fixed on fingers of an user according to an embodiment of the present invention; and

FIG. 9 shows the status a controller is fixed on a probe of an ultrasonic diagnosis detector according to an embodiment of the present invention.

### DETAILED DESCRIPTION OF THE INVENTION

The present invention will be described with reference to the drawings, which present the exemplary embodiments of the present invention. However, embodiments of the present invention can be carried out in other different manners, and should not be understood in such a way that it is confined to these concrete embodiments. In contrast, providing these embodiments is to make the disclosure of the present invention thorough and complete, thereby conveying the conception of the present invention to persons skilled in the art. In the whole text, the same or similar number denotes the same means or unit.

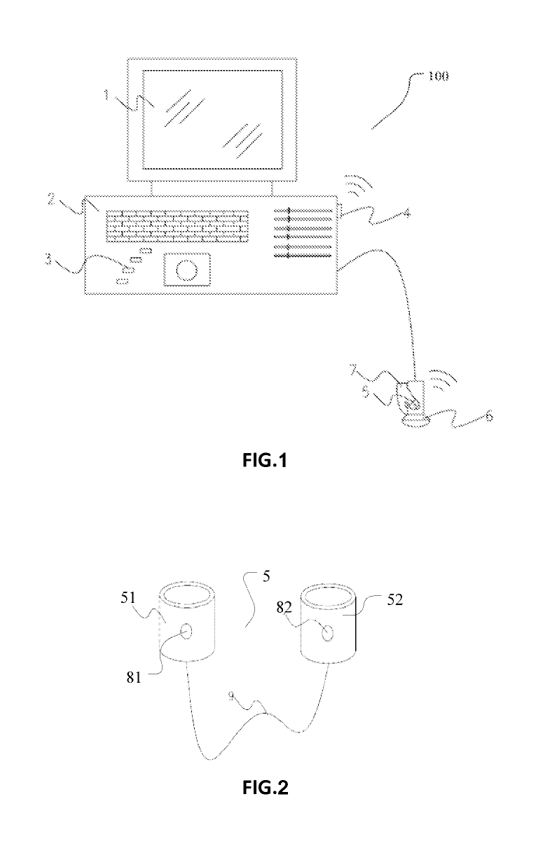

FIG. 1 shows an ultrasonic diagnosis detector 100 according to an embodiment of the present invention. The ultrasonic diagnosis detector 100 comprises an ultrasonic diagnosis detector host machine 2, a main console 3, a probe 6, a display 1, and a controller 5. In virtue of the main console 3, a user controls operations of the ultrasonic diagnosis detector 100. For example, in virtue of the main console 3, the user operates the ultrasonic diagnosis detector host machine 2 so as to emit ultrasonic wave through the probe 6. When impacting an object (for example, an issue within the body of a patient), the ultrasonic wave emitted by the probe 6 is echoed back, thereby forming an echo signal. The ultrasonic diagnosis detector host machine 2 can use the echo signal to generate a corresponding diagnostic image. In an embodiment, the echo signal is captured by the probe 6; after the acoustic-electro conversion, a corresponding electrical signal is generated. In the ultrasonic diagnosis detector host machine 2, the electrical signal is translated into an image to be presented on the display 1, thereby facilitating the real-time observation.

The controller 5 has all the functions of the main console 3 or part of these functions. For example, one could start up and/or shut down the ultrasonic diagnosis detector 100 by the controller 5, or execute various operations of the images displayed on the display by the controller 5, which operations include, but am not limited to freezing an image, scaling an image, adjusting scanning depth, etc. As shown in FIG. 1, the controller 5 can be fixed on a certain part of the body of the user (or an operator). The part may include, but is not limited to, such parts facilitating the user's operations as a finger, a palm or an arm. In particular, when the controller 5 is fixed on the uses finger or palm, the user can operate the probe 6 and the controller 5 conveniently and simultaneously with a single hand. It is easy to understand that when the controller 5 is fixed on the probe 6, one could also operate the probe 6 and the controller 5 simultaneously with a single hand.

FIG. 2 shows a controller 5 according to an embodiment of the present invention. The controller 5 consists of two annular control units 51 and 52, which are connected to each other through a soft cable 9. The two control units 51 and 52 are respectively provided with triggering components 81 and 82 for triggering the corresponding control units to generate control instructions to control operations of the ultrasonic diagnosis detector 100.

It should be noted that the controller S shown herein is illustrative rather than restrictive. According to different circumstances, for example, the controller 5 may also comprise only one control unit or more than two control units, and/or a single control unit may also be provide with more than one triggering components. Although the control units 51 and 52 in FIG. 2 are connected to each other through a soft cable 9, it can be readily understood that a plurality of control units also can be connected with each other in a wireless manner, and/or form a control system network. The control system network can be realized through wireless communication technologies (for example, Bluetooth or ZigBee etc.). These wireless communication technologies are already well-known in the art, and any further elaboration thereon would be only unnecessary.

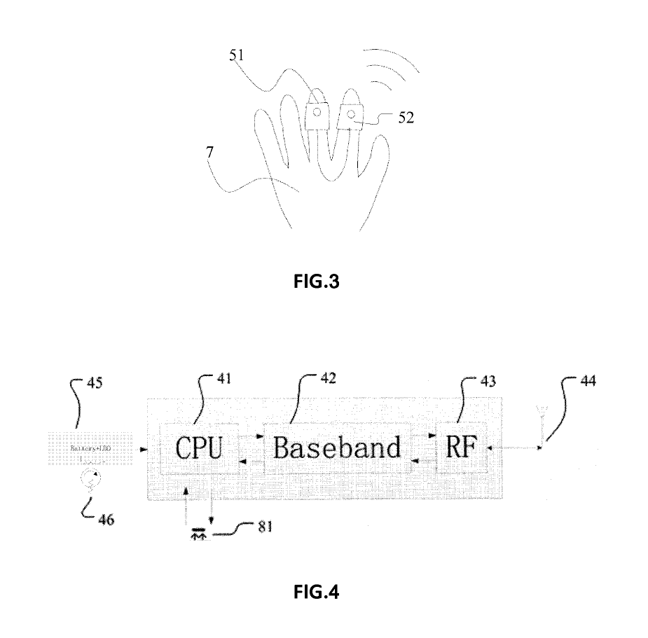

FIG. 3 shows the situation where annular control units of a controller are fixed on fingers of a user 7 according to an embodiment of the present invention. The user can start up (or trigger) the triggering components in various manners, for example, by pushing down the triggering components or pressing the triggering components onto the probe. After the triggering components are started up, the control units generate corresponding control instructions, for example, the instructions for adjusting image displayed on the display. It can be understood that the size of the annular controller also can be made adapted to be fixed on the other parts of a human body or on the probe of the ultrasonic diagnosis detector. For example, as an annular control unit is fixed to a finger, the annular controller also can be put around a palm, an arm or a probe.

The control units of the controller shown in FIGS. 2-3 are the same, but it can be readily understood that the control units also can be different according to different implementations. For example, some of the control units in the controller can be adapted to be fixed on different parts of the body (for example, a finger and/or a palm and/or an arm), while others are adapted to be fixed on a probe or any other adapted components of the ultrasonic diagnosis detector 100.

According to an embodiment, the circuit system of each of the control units of the controller can he at least partially packaged in the housing. The material of the housing includes, but is not limited to, flexible materials, such as rubber. According to an embodiment of the present invention, the control units packaged in the housing of flexible materials are adapted to serve different users (though the size and shape of fingers, palms or arms of different users might differ greatly), or to be fixed onto the probes with different sizes and shapes. According to an embodiment, the housing can further be made of water-proof flexible materials. The solution that the circuit system is packaged in the housing can make it convenient to sanitize and sterilize the control units, and also can improve the durability of the controller. According to an embodiment of the present invention, what are packaged in the housing of each of the control units include a processing unit, a transmitting unit and a power supply module. When the triggering component is triggered, the processing unit will identify different triggering actions and convert them into the corresponding control instructions. For example, different control instructions can be represented in the form of different data frames. The transmitting unit is used to transmit the control instructions in a wired or wireless manner to the ultrasonic diagnosis detector host machine. The power supply module is used to supply energy to the processing unit and the transmitting unit. The power supply module can comprise a button cell battery or other power supplies that are suitable in use and well-known in the art.

FIG. 4 shows the circuit structure of a control unit of a controller according to an embodiment of the present invention, which control unit transmits the control instructions resulted from triggering actions to the ultrasonic diagnosis detector host machine in a wireless manner. Besides the processing unit 41, the transmitting unit and the power supply module 45, the circuit system of the control units further comprises a baseband unit 42 for performing baseband processing on the control instruction generated by the processing unit to obtain a baseband signal carrying the control instruction. Moreover, to meet the requirement for wireless transmission, the transmitting unit further comprises an RF unit 43 and an antenna 44. The RF unit 43 is used to process the baseband signal generated by the baseband unit 42, thereby obtaining the RF signal adapted for wireless transmission through the antenna 44. The wireless communication technology well-known in the art, such as Bluetooth, ZigBee, can be used to transmit the RE signal.

According to an embodiment of the present invention, the power supply module 45 further comprises a battery, an induction coil., and a charging circuit, wherein the charging circuit can charge the battery wirelessly through the induction coil. As such, the external wireless charger 46 can wirelessly charge the power supply module 45 in the control units.

In an embodiment as shown in FIG. 6, the wireless charger comprises a main body 61, a charging pole 11, and a USB interface 10. When charging, the control units are placed on the charging pole 1. Both the charging pole 11 and the control unit 5 are provided with an induction coil therein. The wireless charger obtain electric energy from the ultrasonic diagnosis detector host machine or other devices through the USB interface 10, and after conversion, the electric energy is stored in the batter through the induction coil in the charging pole 11 and the induction coil in the control unit 5.

With the use of the wireless charging solution, the circuit system of the control unit of the controller can be favorably sealed in the water-proof sealed housing. Since medical devices need sanitizing and sterilizing frequently, the solution makes it more convenient to maintain the medial instruments regularly than the traditional power supply solution. In the traditional power supply solution, it is impossible to seal the circuit system in a water-proof sealed housing. For example, for a wired charging power supply and a battery alike, an area that cannot be sealed needs to be preserved in the housing.

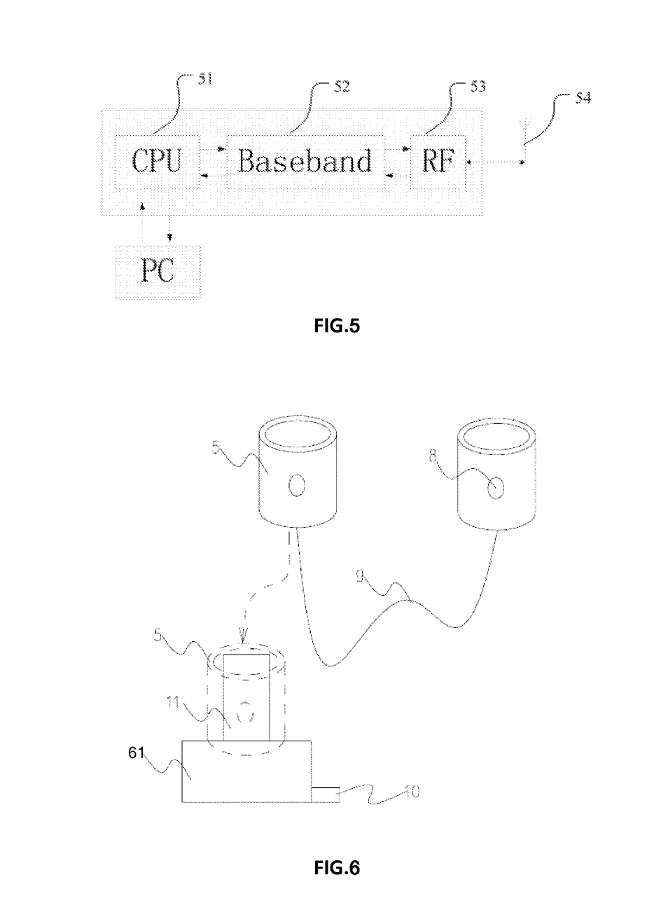

According to an embodiment of the present invention, a wireless unit 4 is attached to the ultrasonic diagnosis detector host machine, as shown in FIG. 1. FIG. 5 shows the circuit structure of a wireless unit on the host machine of an ultrasonic diagnosis detector according to an embodiment of the present invention. Herein, the wireless unit includes, but is not limited to, a wireless unit using a USB interface. The RF unit 53 generates a baseband signal carrying a corresponding instruction from the RF signal received by the antenna 54, and the corresponding control instruction is then regenerated from the baseband signal in the baseband unit 52. The regenerated control instruction corresponds to the instruction generated in the control unit of the controller. Subsequently, the processing unit 51 will translate the corresponding control instruction into a concrete operation that needs executing, and further send it to the main processor PC of the ultrasonic diagnosis detector host machine, in order to execute the concrete operation.

Persons skilled in the art would understand that regenerating the control instruction from the RF signals in the wireless unit 4 is a process opposite to generating the RF signal from the control instruction in the control unit. According to an embodiment of the present invention, the function of transmitting a signal generated by the control unit is also reciprocal to that of receiving a signal by the wireless unit. In other words, the ultrasonic diagnosis detector host machine also can transmit a signal to the controller through the wireless unit. In particular, the ultrasonic diagnosis detector host machine can transmit a signal to a corresponding control unit through the wireless unit so as to instruct the indicating means arranged in the control unit to show a user the current working status of the controller (or the status of the ultrasonic diagnosis detector). For example, it can provide the user with a warning that the configuration of the current controller is not correct. The indicating means includes, but is not limited to, an indicator light and/or a vibrator.

According to an embodiment, when needing to use the controller of the present invention, a user does not need to replace the old host machine or probe with a new host machine or probe. In contrast, the user only needs to select a proper controller and wireless unit. In particular, the user only needs to fix the control unit of the controller onto appropriate positions (for example, a finger, a palm, an arm or a probe), connect the wireless unit (for example, through a USB interface) to the ultrasonic diagnosis detector host machine, and correspondingly install a control driver program in the host machine. Moreover, when one or more control units of the controller is or are damaged, one only needs to replace the damaged control unit(s) instead of the whole controller.

In an embodiment of the present invention, when a control unit pairs with a wireless unit, the user presses and holds down the button on the wireless unit until the indicator light on the wireless unit flickers, and simultaneously presses and holds down the triggering component (for example, a button) on one control unit of the controller until the indicator light on the control unit flickers. When the two indicator lights stop flickering and are constantly alight, the pairing between the control unit and the wireless unit provided on the host machine is accomplished. Likewise, the pairings between a plurality of control units and the wireless unit can be accomplished.

According to an embodiment of the present invention, one or more triggering components of the controller can be configured in such a way that a single triggering component or the combination of a plurality of triggering, components can be used to trigger different control instructions. Take the two control units that respectively have one triggering component for example, the click of the first triggering component is to zoom out an image, the click of the second triggering component is to zoom down an image, and simultaneous click of the first and second triggering component is to freeze or unfreeze an image, long press of the first triggering component and click of the second triggering component is to increase or decrease focusing depth, long press of the second triggering component is to adjust the mage contrast, etc. In the host machine, the user can define operations triggered by the control units through software on his/her own initiative. Such means as a LED light or a vibrator arranged on the control unit also can be used to provide feedback of triggering actions.

In addition, although what is introduced in details in the embodiments is the wireless communication between the controller and the ultrasonic diagnosis detector host machine, it is easy to understand that the communication also can be carried out in a wired manner. These wireless or wired communication technologies are already well-known in the art, and any further elaboration thereon would be only unnecessary.

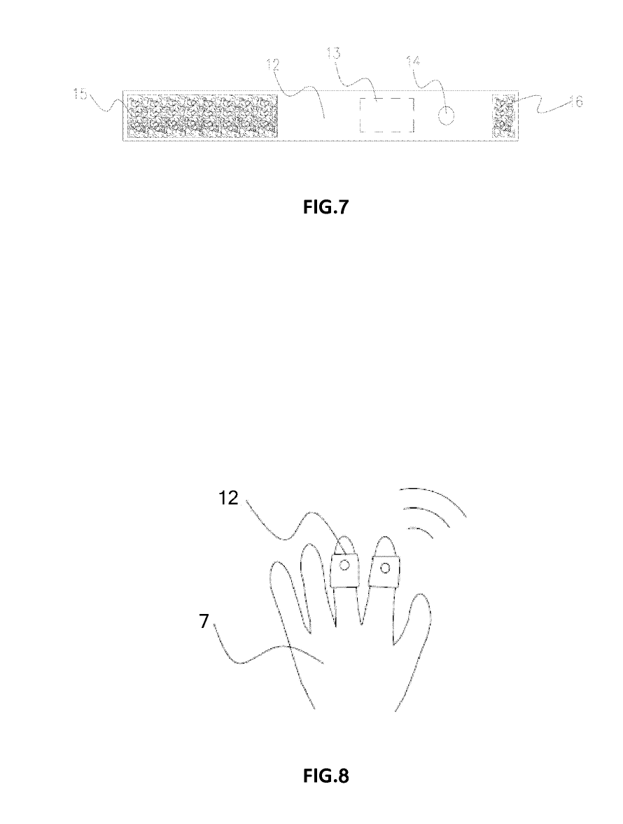

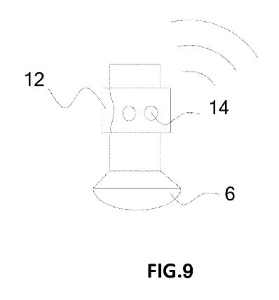

FIG. 7 shows the unfolding status of a control unit of a controller according to an embodiment of the present invention. As shown in the figure, the control unit is a belt-like control unit 12, comprising joint components 15 and 16, a triggering component 14 and a circuit system 13 arranged within the control unit. The belt-like control unit 12 is adapted to be wound (or bound) around (or to) a portion of a body and/or a probe of an ultrasonic diagnosis detector, as shown in FIGS. 8 and 9. This can be accomplished by engaging the joint components 15 and at the two ends of the belt-like control unit 12 to form a ring in a suitable size. The joint components can make it easy to engage or separate the two ends of the belt-like control unit 12. Moreover, the joint manner includes, but is not limited to, self-adhesive band, magnetic bond, etc. Except for the fixing manner, the belt-like control unit is similar to the ring-shaped control unit introduced above in other aspects.

Although the above disclosure in combination with the ultrasonic diagnosis detector sets forth embodiments of the present invention in details, persons skilled in the art can understand that the conception of the present invention is also applicable to control other apparatuses and devices. For example, the controller of an embodiment of the present invention also can be used to control machines in production departments, electrical toys, household electric appliances, etc. In accordance with an embodiment of the present invention, the controller together with other apparatuses and devices can form a system which is adaptable to be controlled by the controller.

In addition, though some particular terms are used in the description, they are only illustrative. There further exist many other embodiments without departing from the spirit and scope of the present invention, all of which fall within the protection scope claimed by the present application. The protection scope of the present invention is defined by the appended claims.

Embodiments of the present invention aim to solve one or more problems existing in the prior art.

According to at least one embodiment of the present invention, a controller is provided for controlling operations of an ultrasonic diagnosis detector. The controller comprises one or more control units, and one or more triggering components arranged on the one or more control units. The one or more triggering components are used for triggering the one or more control units to generate a corresponding control instruction, thereby controlling operations of the ultrasonic diagnosis detector. At least one of the one or more control units is configured to be fixed on a portion of a body and/or a probe of the ultrasonic diagnosis detector.

In at least one embodiment, the portion of the body is a finger, a palm or an arm of a user, and the control of operations of the ultrasonic diagnosis detector includes the control of image display of the ultrasonic diagnosis detector.

In at least one embodiment, at least one of the one or more control units is an annular control unit, which is adapted to be put on the portion of the body and/or placed around the probe of the ultrasonic diagnosis detector.

In at least one embodiment, at least one of the one or more control units is a belt-like control unit, which is adapted to he wound around the portion of the body and/or the probe of the ultrasonic diagnosis detector.

In at least one embodiment, each of at least one of the one or more control units comprises a processing unit for generating a corresponding control instruction when the one or more triggering components are triggered, a transmitting unit for transmitting the corresponding control instruction to a host machine of the ultrasonic diagnosis detector, a power supply module for supplying energy to the processing unit and the transmitting unit.

In at least one embodiment, each of at least one of the one or more control units further comprises, a baseband unit for performing a baseband processing on the corresponding control instruction generated by the processing unit to obtain a baseband signal, which carries the corresponding control instruction, the transmitting unit further comprises an RF unit for processing the baseband signal to obtain an RF signal, and an antenna for transmitting the RF signal to the host machine of the ultrasonic diagnosis detector.

In at least one embodiment, the power supply module comprises a battery, an induction coil, a charging circuit for charging the battery wirelessly by the induction coil.

In at least one embodiment, the one or more triggering components can be configured in such a way that a single triggering component or the combination of a plurality of triggering components triggers different control instructions.

In at least one embodiment, at least a part of the one or more control units can communicate mutually or form a control system network.

In at least one embodiment, at least one of the one or more control units is provided with an indicating means for indicating the working status of the controller.

In at least one embodiment, the indicating means comprises an indicator light and/or a vibrator.

In at least one embodiment, each of at least one of the one or more control units is provided with more than one triggering components.

In at least one embodiment, at least a part of the one or more triggering components is a button.

In at least one embodiment, the one or more control units can be replaced individually.

In at least one embodiment, each of at least one of the one or more control units comprises a housing and at least a part of the processing unit, the transmitting unit and the power supply module is packaged in the housing, which is made of a flexible material.

According to at least one embodiment of the present invention, an ultrasonic diagnosis detector is provided. The detector comprises an ultrasonic diagnosis detector host machine, a probe, and a console for controlling the ultrasonic diagnosis detector host machine so as to emit ultrasonic wave through the probe, wherein the ultrasonic diagnosis detector host machine generates an image based on an echo signal of the ultrasonic wave. The detector further composes a display for displaying the image, and a controller for controlling operations of the ultrasonic diagnosis detector, wherein the controller is adapted to be fixed on a portion of a body and/or the probe.

In at least one embodiment, the controller comprises: one or more control units, one or more triggering components arranged on the one or more control units, the one or more triggering components used for triggering the one or more control units to generate a corresponding control instruction, thereby controlling operations of the ultrasonic diagnosis detector, wherein at least one of the one or more control units is adapted to be fixed on a portion of a body and/or the probe.

In at least one embodiment, the portion of the body is a linger, a palm or an arm of a user, and the control of operations of the ultrasonic diagnosis detector includes the control of image display of the ultrasonic diagnosis detector.

In at least one embodiment, at least one of the one or more control units is an annular control unit, which is adapted to be put on the portion of the body and/or placed around the probe of the ultrasonic diagnosis detector.

In at least one embodiment, at least one of the one or more control units is a belt-like control unit, which is adapted to he wound around the portion of the body and/or the probe of the ultrasonic diagnosis detector.

In at least one embodiment, each of at least one of the one or more control units comprises a processing unit for generating a corresponding control instruction when the one or more triggering components are triggered, a transmitting unit for transmitting the corresponding control instruction to a host machine of the ultrasonic diagnosis detector, a power supply module for supplying energy to the processing unit and the transmitting unit.

In at least one embodiment, each of at least one of the one or more control units further comprises a baseband unit for performing a baseband processing on the corresponding control instruction generated by the processing unit to obtain a baseband signal, which carries the corresponding control instruction; the transmitting unit further comprises an RF unit for processing the baseband signal to obtain an RF signal, and an antenna for transmitting the RF signal to the host machine of the ultrasonic diagnosis detector.

In at least one embodiment, the ultrasonic diagnosis detector host machine is coupled to a wireless unit for receiving the RF signal and regenerating a control instruction from the RF signal to control operations of the ultrasonic diagnosis detector.

In at least one embodiment, the power supply module comprises a battery, an induction coil, a charging circuit for charging the battery wirelessly by the induction coil.

In at least one embodiment, the one or more triggering components can be configured in such a way that a single triggering component or the combination of a plurality of triggering components triggers different control instructions.

In at least one embodiment, at least a part of the one or more control units can communicate mutually or form a control system network.

In at least one embodiment, at least one of the one or more control units is provided with an indicating means for indicating working status of the controller.

In at least one embodiment, the indicating means comprises an indicator light and/or a vibrator.

In at least one embodiment, each of at least one of the one or more control units is provided with one or more triggering components.

In at least one embodiment, at least a part of the one or more triggering components is a button.

In at least one embodiment, the one or more control units can be individually replaced.

In at least one embodiment, each of at least one of the one or more control units comprises a housing and at least a part of the processing unit, the transmitting unit and the power supply module are packaged in the housing, which is made of a flexible material.

According to at least one embodiment of the present invention, a controller is provided, which comprises one or more control units, and one or more triggering components arranged on the one or more control units. The one or more triggering component are used for triggering the one or more control units to generate a corresponding control instruction, wherein at least one of the one or more control units is configured to be fixed on a portion of a body.

In at least one embodiment, the portion of the body is a finger, a palm or an arm of a user.

In at least one embodiment, at least one of the one or more control units is an annular control unit, which is adapted to be put on the portion of the body.

In at least one embodiment, at least one of the one or more control units is a belt-like control unit, which is adapted to be wound around the portion of the body.

In at least one embodiment, the controller comprises a wireless transmitter for transmitting a corresponding control instruction wirelessly.

In at least one embodiment, the controller comprises a power supply module including a battery, an induction coil, and a charging circuit for charging the battery wirelessly by the induction coil.

In at least one embodiment, the one or more triggering components can be configured in such a way that a single triggering component or the combination of a plurality of triggering components triggers different control instructions.

According to at least one embodiment of the present invention, a system is provided that controls a device by using a controller which comprises one or more control units and one or more triggering components arranged on the one or more control units. The one or more triggering components are used for triggering the one or more control units to generate a corresponding control instruction, wherein at least one of the one or more control units is adapted to be fixed on a portion of a body.

The controllers of the present invention can be manufactured and replaced in a simple way, and a user can control operations of the ultrasonic diagnosis detector flexibly, quickly and conveniently with the controller of the present invention.

What is claimed is:

1. A controller for controlling operations of an ultrasonic diagnosis detector, comprising:
one or more control units; and
one or more triggering components arranged on the one or more control units,
wherein the one or more triggering components are configured to trigger the one or more control units to generate a corresponding control instruction to control operations of the ultrasonic diagnosis detector, wherein at least one of the one or more control units is configured to be fixed on a portion of a body and/or a probe of the ultrasonic diagnosis detector.

2. The controller according to claim 1, wherein the portion of the body is a finger, a palm or an arm of an user, and the operations of the ultrasonic diagnosis detector controlled by the one or more triggering components comprise controlling image display of the ultrasonic diagnosis detector.

3. The controller according to claim 2, wherein at least one of the one or more control units is an annular control unit, and is configured to be put on the portion of the body and/or be placed around the probe of the ultrasonic diagnosis detector.

4. The controller according to claim 2, wherein at least one of the one or more control units is a belt-like control unit, and is configured to be wound around the portion of the body and/or the probe of the ultrasonic diagnosis detector.

5. The controller according to claim 1, wherein at least one of the one or more control units comprises:
a processing unit configured to generate a corresponding control instruction when the one or more triggering components are triggered;
a transmitting unit configured to transmit the corresponding control instruction to a host machine of the ultrasonic diagnosis detector; and
a power supply module configured to supply energy to the processing unit and the transmitting unit.

6. The controller according to claim 5, wherein at least one of the one or more control units further comprises:
a baseband unit configured to perform a baseband processing on the corresponding control instruction generated by the processing unit to obtain a baseband signal, which carries the corresponding control instruction, and
the transmitting unit further comprises:
an RF unit configured to process the baseband signal to obtain an RF signal; and
an antenna configured to transmit the RF signal to the host machine of the ultrasonic diagnosis detector.

7. The controller according to claim 5, wherein the power supply module comprises:
a battery;
an induction coil; and
a charging circuit for wirelessly charging the battery through the induction coil.

8. The controller according to claim 1, wherein the one or more triggering components are configured in such a way that a single triggering component or a combination of a plurality of triggering components triggers different control instructions.

9. The controller according to claim 1, wherein at least a part of the one or more control units communicates with each other or forms a control system network.

10. The controller according to claim 1, wherein at least one of the one or more control units is provided with an indicator configured to indicate a working status of the controller.

11. The controller according to claim 10, wherein the indicator comprises an indicator light and/or a vibrator.

12. The controller according to claim 1, wherein at least one of the one or more control units is provided with one or more triggering components.

13. The controller according to claim 1, wherein at least one of the one or more triggering components is a button.

14. The controller according to claim 1, wherein the one or more control units is individually replaceable.

15. The controller according to claim 5, wherein at least one of the one or more control units comprises a housing made of a flexible material, and at least a part of the processing unit, the transmitting unit and the power supply module is sealed in the housing.

16. An ultrasonic diagnosis detector, comprising:
an ultrasonic diagnosis detector host machine;
a probe;
a console configured to control the ultrasonic diagnosis detector host machine so as to emit ultrasonic wave through the probe, wherein the ultrasonic diagnosis detector host machine generates an image based on an echo signal of the ultrasonic wave;
a display configured to display the image; and
a controller configured to control operations of the ultrasonic diagnosis detector;
wherein the controller is configured to be fixed on a portion of a body and/or the probe.

17. The ultrasonic diagnosis detector according to claim 16, wherein the controller comprises:
one or more control units;
one or more triggering components arranged on the one or more control units,
wherein the one or more triggering components are configured to trigger one or more control units to generate a corresponding control instruction to control operations of the ultrasonic diagnosis detector, wherein at least one of the one or more control units is configured to be fixed on a portion of a body and/or the probe.

18. The ultrasonic diagnosis detector according to claim 17, wherein the portion of the body is a finger, a palm or an arm of a user, and the operations of the ultrasonic diagnosis detector controlled by the one or more triggering components comprise controlling image display of the ultrasonic diagnosis detector.

19. The ultrasonic diagnosis detector according to claim 18, wherein at least one of the one or more control units is an annular control unit, and is configured to be put on the portion of the body and/or placed around the probe of the ultrasonic diagnosis detector.

20. The ultrasonic diagnosis detector according to claim 18, wherein at least one of the one or more control units is a belt-like control unit, and is configured to be wound around the portion of the body and/or the probe of the ultrasonic diagnosis detector.

21. The ultrasonic diagnosis detector according to claim 16, wherein at least one of the one or more control units comprises:
a processing unit configured to generate a corresponding control instruction when the one or more triggering components are triggered;
a transmitting unit configured to transmit the corresponding control instruction to the ultrasonic diagnosis detector host machine; and
a power supply module configured to supply energy to the processing unit and the transmitting unit.

22. The ultrasonic diagnosis detector according to claim 21, wherein at least one of the one or more control units further comprises:
a baseband unit configured to perform a baseband processing on the corresponding control instruction generated by the processing unit to obtain a baseband signal, which carries the corresponding control instruction, and
the transmitting unit further comprises:
an RF unit configured to process the baseband signal to obtain an RF signal; and
an antenna configured to transmit the RF signal to the ultrasonic diagnosis detector host machine.

23. The ultrasonic diagnosis detector according to claim 22, wherein the ultrasonic diagnosis detector host machine is coupled to a wireless unit configured to receive the RF signal and to regenerate the control instruction from the RF signal to control operations of the ultrasonic diagnosis detector.

24. The ultrasonic diagnosis detector according to claim 21, wherein the power supply module comprises:
a battery;
an induction coil; and
a charging circuit configured to wirelessly charge the battery by the induction coil.

25. The ultrasonic diagnosis detector according to claim 16, wherein the one or more triggering components is configured in such a way that a single triggering component or a combination of a plurality of triggering components triggers different control instructions.

26. The ultrasonic diagnosis detector according to claim 16, wherein at least a part of the one or more control units communicates with each other or forms a control system network.

27. The ultrasonic diagnosis detector according to claim 16, wherein at least one of the one or more control units is provided with an indicator configured to indicate a working status of the controller.

28. The ultrasonic diagnosis detector according to claim 16, wherein the indicator comprises an indicator light and/or a vibrator.

29. The ultrasonic diagnosis detector according to claim 16, wherein at least one of the one or more control units is provided with one or more triggering components.

30. The ultrasonic diagnosis detector according to claim 16, wherein at least one of the one or more triggering components is a button.

31. The ultrasonic diagnosis detector according to claim 16, wherein the one or more control units is individually replaceable.

32. The ultrasonic diagnosis detector according to claim 21, wherein at least one of the one or more control units comprises a housing made of a flexible material, and at least a part of the processing unit, the transmitting unit and the power supply module is packaged in the housing.

33. A controller, comprising:
one or more control units; and
one or more triggering components arranged on the one or more control units, wherein the one or more triggering components is configured to trigger the one or more control units to generate a corresponding control instruction,
wherein at least one of the one or in more control units is configured to be fixed on a portion of a body.

34. The controller according to claim 33, wherein the portion of the body is a finger, a palm or an arm of a user.

35. The controller according to claim 34, wherein at least one of the one or more control units is an annular control unit, which is configured to be put on the portion of the body.

36. The controller according to claim 34, wherein at least one of the one or more control units is a belt-like control unit, and is configured to be wound around the portion of the body.

37. The controller according to claim 34, wherein the controller comprises a wireless transmitter configured to wirelessly transmit a corresponding control instruction.

38. The controller according to claim 33, wherein the controller comprises a power supply module, wherein the power supply module comprises:
a battery;
an induction coil; and
a charging circuit configured to wirelessly charge the battery by the induction coil.

39. The controller according to claim 33, wherein the one or more triggering components is configured in such a way that a single triggering component or a combination of a plurality of triggering components triggers different control instructions.
